(19) United States
(12) Patent Application Publication
(10) Pub. No.: US 2025/0360301 A1
(43) Pub. Date: Nov. 27, 2025

(54) MECHANICAL CIRCULATORY SUPPORT PUMP CLIP AND CORD MANAGEMENT SYSTEM

(71) Applicant: BOSTON SCIENTIFIC SCIMED, INC., Maple Grove, MN (US)

(72) Inventors: Kimberly Anne Robertson, Forest Lake, MN (US); Lloyd Radman, Blaine, MN (US); Jeffrey Matthew Lucas, Eden Prairie, MN (US); Brett Struthers, Byron, MN (US)

(73) Assignee: BOSTON SCIENTIFIC SCIMED, INC., Maple Grove, MN (US)

(21) Appl. No.: 19/216,012

(22) Filed: May 22, 2025

Related U.S. Application Data
(60) Provisional application No. 63/651,209, filed on May 23, 2024.

(51) Int. Cl. A61M 60/13; A61M 25/00; A61M 25/02; A61M 60/216

(52) U.S. Cl. CPC A61M 60/13; A61M 60/216; A61M 25/0097; A61M 2025/024

(57) ABSTRACT

Mechanical circulatory support devices with cord management systems are disclosed. An example percutaneous circulatory support device comprises a catheter including an elongate shaft and a percutaneous blood pump positioned at a distal end of the elongate shaft. A junction box is secured to the elongate shaft proximal of the percutaneous blood pump, and a clip assembly is disposed on an outer surface of the junction box.

### CROSS REFERENCE TO RELATED APPLICATIONS

The present application claims the benefit of U.S. Provisional Patent Application Ser. No. 63/651,209, filed May 23, 2024, the disclosure of which is incorporated herein by reference.

### TECHNICAL FIELD

The present disclosure pertains to a catheter including a percutaneous blood pump. More particularly, the present disclosure pertains to a mechanical circulatory support device having a cord management system.

### BACKGROUND

Percutaneous mechanical circulatory support devices, such as blood pumps can provide transient support for hours, days or months of use in patients whose heart function or cardiac output is compromised. The percutaneous mechanical circulatory support devices may be sufficiently flexible to be navigated through the vasculature to a patient's heart. For instance, such devices may be delivered percutaneously from the femoral artery, retrograde through the descending aorta, over the aortic arch, through the ascending aorta across the aortic valve, and into the left ventricle.

To assist a clinician using a circulatory support device during a medical procedure, a circulatory support device system may include one or more junction boxes along a catheter for containing electrical connections, capacitor board, memory card, batteries, limited motor controllers, optical fiber connections, and the like. However, there is an ongoing need to provide circulatory support device systems including support pump clip and cord management systems which may prevent movement of the catheter and blood pump while inserted into a patient, as well as prevent cords from falling out of the sterile field.

### SUMMARY

This disclosure provides design, material, manufacturing method, and use alternatives for medical devices. An example medical device includes a percutaneous circulatory support device comprising, a catheter including an elongate shaft and a percutaneous blood pump positioned at a distal end of the elongate shaft, a junction box having a proximal end and a distal end, wherein the junction box is secured to the elongate shaft proximal of the percutaneous blood pump such that the elongate shaft extends between the junction box and the percutaneous blood pump, and a clip assembly disposed on an outer surface of the junction box.

Alternatively or additionally to the embodiment above, the clip assembly further comprises a base end portion attached to and extending radially outward from the outer surface of the junction box, a free end portion biased to touch the outer surface of the junction box, and a central portion extending between the base end portion and the free end portion.

Alternatively or additionally to the embodiment above, the base end portion comprises a preformed curved portion.

Alternatively or additionally to the embodiment above, the clip assembly is comprised of a flexible material.

Alternatively or additionally to the embodiment above, the junction box further comprises a recess along the outer surface of the junction box, disposed under the free end portion of the clip assembly.

Alternatively or additionally to the embodiment above, the clip assembly includes a bump configured to nest within the recess of the junction box.

Alternatively or additionally to the embodiment above, the central portion of the clip assembly is spaced apart from the junction box defining a space between the outer surface of the junction box and the central portion of the clip assembly.

Alternatively or additionally to the embodiment above, the clip assembly is configured to retain the elongate shaft within the space between the outer surface of the junction box and central portion of the clip assembly.

Alternatively or additionally to the embodiment above, the clip assembly is configured to retain a bed sheet, a drape, a covering, a gown, a strap, a pole, or a bed rail within the space between the outer surface of the junction box and the central portion of the clip assembly.

Alternatively or additionally to the embodiment above, the junction box further includes a channel disposed on the outer surface of the junction box.

Alternatively or additionally to the embodiment above, the clip assembly is disposed within the channel.

Alternatively or additionally to the embodiment above, a free end portion of the clip assembly is configured to curve or extend radially outward at an angle from the junction box beyond the channel.

Alternatively or additionally to the embodiment above, the clip assembly further comprises a central portion pivotably coupled to the junction box, a first end portion extending from the central portion, a second end portion extending from the central portion opposite the first end portion, and a spring biasing the second end portion against the outer surface of the junction box.

Alternatively or additionally to the embodiment above, the first end portion is spaced away from the outer surface of the junction box when the second end portion is urged against the outer surface of the junction box.

Alternatively or additionally to the embodiment above, the second end portion is configured to be moved away from the outer surface of the junction box as the first end portion is moved toward the outer surface of the junction box.

An example percutaneous circulatory support device includes a catheter including an elongate shaft and a percutaneous blood pump positioned at a distal end of the elongate shaft, a junction box having a proximal end and a distal end, wherein the elongate shaft extends distally from the distal end of the junction box to the percutaneous blood pump, a clip assembly disposed on an outer surface of the junction box, the clip assembly comprising a base end portion attached to and extending radially outward from the outer surface of the junction box, a free end portion biased to touch the outer surface of the junction box, and a central portion extending between the base end portion and the free end portion, wherein the clip assembly is comprised of a flexible material such that the free end portion can be deflected away from the outer surface of the junction box.

Alternatively, or additionally to any of the embodiments above, the junction box further comprises a recess along the outer surface of the junction box, wherein the free end portion of the clip assembly extends into the recess.

Alternatively, or additionally to any of the embodiments above, the clip assembly is spaced apart from the junction box between the base end portion and the free end portion to define a space therebetween, wherein the clip assembly is configured to retain the elongate shaft within the space between the junction box and the clip assembly.

An example percutaneous circulatory support device includes a catheter including an elongate shaft and a percutaneous blood pump positioned at a distal end of the elongate shaft, a junction box having a proximal end and a distal end, wherein the elongate shaft extends distally from the distal end of the junction box to the percutaneous blood pump, a clip assembly pivotably coupled to the junction box, wherein the clip assembly comprises, a central portion pivotably coupled to the junction box, a first end portion extending from the central portion, a second end portion extending from the central portion opposite the first end portion, a spring biasing the second end portion against the outer surface of the junction box.

The above summary of some embodiments, aspects, and/or examples is not intended to describe each embodiment or every implementation of the present disclosure. The figures and the detailed description which follows more particularly exemplify these embodiments.

### BRIEF SUMMARY

This disclosure provides design, material, manufacturing method, and use alternatives for medical devices, including percutaneous circulatory support devices and associated percutaneous blood pumps.

### BRIEF DESCRIPTION OF THE DRAWINGS

The disclosure may be more completely understood in consideration of the following detailed description in connection with the accompanying drawings, in which:

FIG. 1 is a perspective view of an exemplary percutaneous circulatory support device including a percutaneous blood pump and a junction box.

FIG. 2 shows the distal end region of the percutaneous circulatory support device of FIG. 1 including the percutaneous blood pump.

FIG. 3 is a perspective view of an example junction box.

FIG. 4 is a perspective view of an example junction box.

FIG. 5 is a perspective view of an example junction box.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and case of understanding, while every feature and/or element may not be shown in each drawing, the feature(s) and/or element(s) may be understood to be present regardless, unless otherwise specified.

Current mechanical circulation support pumps require a junction box for containing electrical connections along the length of the catheter. The nature of the junction box may affect movement of the catheter, causing a portion of the catheter to fall out of the sterile field, which may pull a pump or circulatory support device out of position. This increases risk of patient harm, and can add time and complication to a procedure. Even without a junction box, the cords and catheters of a circulatory support device can be difficult to manage and keep within the sterile field.

Accordingly, this disclosure is directed to percutaneous circulatory support devices and percutaneous circulatory support systems that include a clip and cord management system. Notably, the junction box may be clipped in place to a strap, bedpost or other stationary item. Additionally, the cord and catheter may be wound or coiled through the clip.

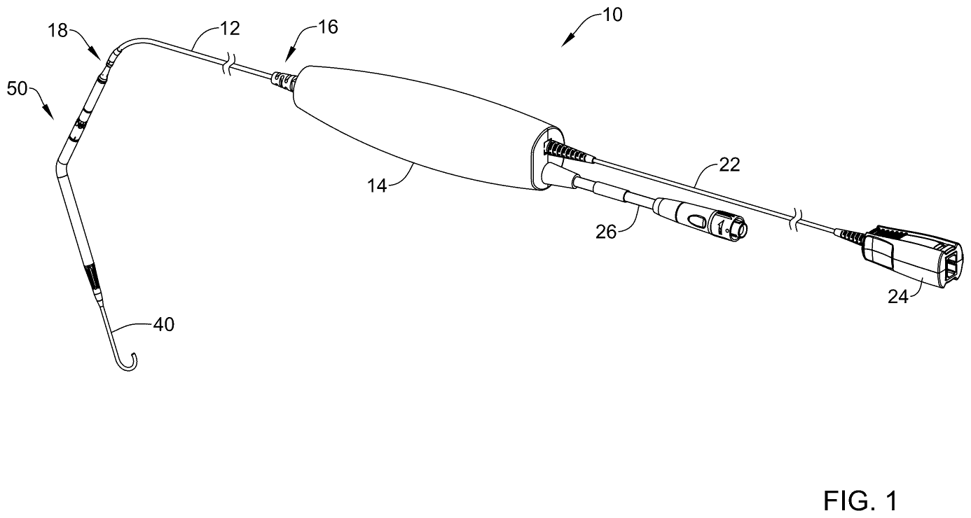

FIG. 1 is a perspective view of an exemplary percutaneous circulatory support device (e.g., catheter) 10 including a percutaneous blood pump 50 with a lactate sensor (not shown in FIG. 1). As illustrated in FIG. 1, the percutaneous blood pump 50 may be located at a distal end region of the catheter 10. The catheter 10 may be coupled to or include the percutaneous blood pump 50, with an elongate shaft 12 of the catheter 10 extending proximally from the percutaneous blood pump 50 and a distal tip 40 extending distally from the percutaneous blood pump 50. For instance, a proximal end 16 of the elongate shaft 12 may be coupled to a junction box 14 and a distal end 18 of the elongate shaft 12 may be coupled to the percutaneous blood pump 50. An electrical cable or optical fiber 22 may extend from the junction box 14 to a connector 24 at a proximal end thereof. The connector 24 may be configured to be connected to a controller or console (not shown in FIG. 1) for controlling the percutaneous blood pump 50, such as providing electrical power to the blood pump 50 or receiving pressure sensor information, or information from another sensor, from the blood pump 50. The catheter 10 may also include an extension 26 connectable to the controller or console (not shown in FIG. 1) for sending and/or receiving signals, such as from one or more sensors during operation of the percutaneous blood pump 50. In some instances, the electrical power cable and/or sensor cables may be combined in a single cable harness, if desired. The junction box 14 may include electrical motor wires running through the shaft 12, which are crimped or soldered to the cable 22 or crimped or soldered to a circuit board connected to the cable 22, providing electrical power to the motor from an electrical source connected to the cable 26. Fiber optics from the catheter shaft 12 may also be fused to fiber optic cables within the junction box 14. Connections between fiber optics may be made with fiber optic ferrell connectors or any other method desired.

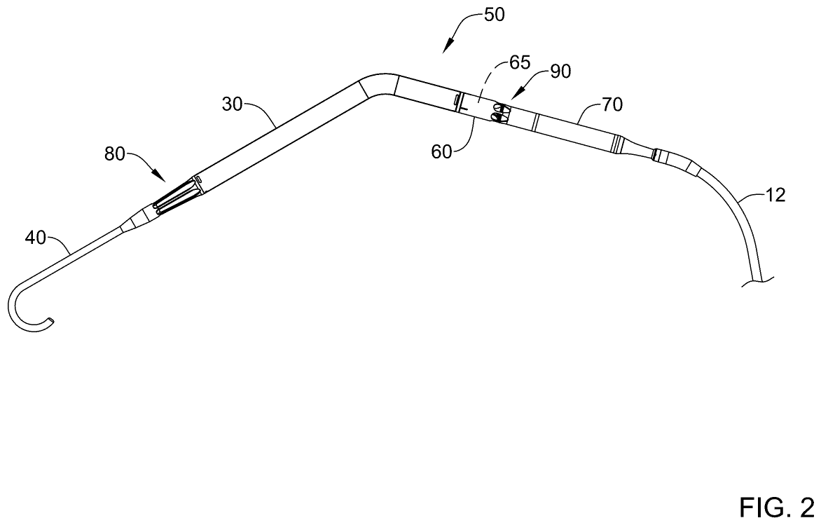

Additional features of the percutaneous blood pump 50 are illustrated in FIG. 2. The percutaneous blood pump 50 may generally include a flexible cannula 30, an impeller housing 60, and a motor housing 70. In some embodiments, the flexible cannula 30, the impeller housing 60 and/or the motor housing 70 may be integrally or monolithically constructed. In other instances, the flexible cannula 30, the impeller housing 60 and/or the motor housing 70 may be separate components. The impeller housing 60 carries an impeller assembly 65 therein. The impeller assembly 65 may include an impeller secured to an impeller shaft that rotates relative to the impeller housing 60 to drive blood through the blood pump 50. In some embodiments, the impeller shaft and the impeller of the impeller assembly 65 may be integrally formed, whereas, in other embodiments the impeller shaft and the impeller may be separate components.

Rotation of the impeller causes blood to flow from a blood inlet 80 of the percutaneous blood pump 50, such as at a distal end of the flexible cannula 30, through the flexible cannula 30 and the impeller housing 60, and out of a blood outlet 90 proximal of the impeller, such as through a sidewall formed on the impeller housing 60. In some instances, the blood inlet 80 may include a plurality of blood inlet windows arranged around a circumference of the percutaneous blood pump 50 (e.g., the flexible cannula 30). In some instances, the blood outlet 90 may include a plurality of blood outflow windows arranged around a circumference of the impeller housing 60. In other embodiments, the blood inlet 80 and/or the blood outlet 90 may be formed on other portions of the percutaneous blood pump 50.

With continued reference to FIG. 2, the motor housing 70 carries a motor configured to rotatably drive the impeller of the impeller assembly 65 relative to the impeller housing 60. Electrical power may be supplied to the motor through wiring extending through the elongate shaft 12, for example. In some instances, the motor may be physically connected to the impeller. For example, in some embodiments the impeller may be mounted on the drive shaft of the motor. In other embodiments, the impeller shaft may be directly or indirectly coupled to the drive shaft of the motor. In some instances, the drive assembly may include a magnetic coupling between the motor and the impeller. For example, a driving magnet may be mounted on the drive shaft of the motor. Rotation of the driving magnet causes rotation of a driven magnet, which is connected to the impeller assembly 65. More specifically, in embodiments incorporating an impeller shaft, the impeller shaft and the impeller of the impeller assembly 65 are configured to rotate with the driven magnet. In other embodiments, the motor may be coupled to the impeller assembly 65 via other components.

In some embodiments, the system herein includes a sensor provided with the percutaneous circulatory support device 10 and/or provided with an introducer sheath configured to be used with introducing and/or maintaining the percutaneous circulatory support device 10 in a patient. Accordingly, in some embodiments, the sensor can be located on an exterior (e.g., outer) surface of the percutaneous circulatory support device 10 (e.g., an exterior surface of the elongate shaft 12, an exterior surface of the cannula 30, an exterior surface of the motor housing 70, or an exterior surface of the impeller housing 60) and/or on an exterior surface of an introducer sheath, as described herein. However, in some embodiments the sensors herein can be configured (e.g., sized, shaped, etc.) to be disposed within a lumen (e.g., on an inner surface of the cannula 30) of the percutaneous circulatory support device 10 or within a lumen of an introducer sheath, as described herein.

In some instances, the sensor can be non-removably coupled to a surface such as an exterior surface of the percutaneous circulatory support device 10 (e.g., an exterior surface of the elongate shaft 12, an exterior surface of the cannula 30, an exterior surface of the motor housing 70, or an exterior surface of the impeller housing 60) or an inner surface of a lumen of the percutaneous blood pump 50 (e.g., the lumen of the cannula 30). Having the lactate sensor be non-removably coupled to the surface can ensure that the sensor does not inadvertently become dislodged from the surface (e.g., an inner surface or exterior surface of the percutaneous circulatory support device 10) and/or may promote other aspects herein such as providing an accurate in-vivo measurement. However, in some instances, the sensor can be removably coupled to a surface such as an exterior surface of the percutaneous circulatory support device 10 (e.g., an exterior surface of the elongate shaft 12, an exterior surface of the cannula 30, an exterior surface of the motor housing 70, or an exterior surface of the impeller housing 60) or an inner surface of a lumen of the percutaneous blood pump 50 (e.g., the lumen of the cannula 30). Having the sensor be removably coupled to the surface can permit the sensor to be readily changed or cleaned (e.g., responsive to a malfunction of a lactate sensor, fouling of the lactate sensor, etc.), and thereby can promote aspects herein such as providing accurate in-vivo measurements.

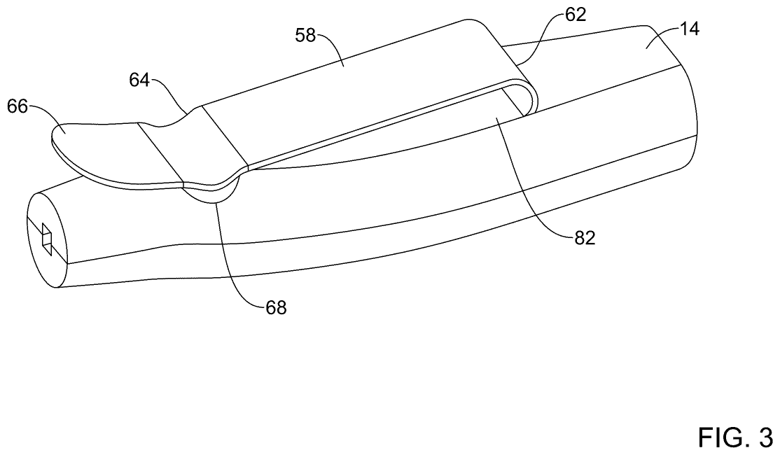

FIG. 3 is a perspective view of a junction box 14 as shown in FIG. 1. In some embodiments the junction box 14 may have a clip 58. The clip 58 and junction box 14 may be a single monolithic element, or they may be separate pieces attached to each other. In general, the clip 58 may be comprised of a flexible material being biased toward a closed position, wherein both first and second ends of the clip assembly are joined to, in contact with, or touching the outer surface of the junction box 14. The clip 58 may include a first curved portion 62 which is connected to (e.g., attached to or continuous with) the junction box 14, and second, open end 66 which is disconnected from the junction box 14, and able to be deflected away from the outer surface of the junction box 14 and urged against the outer surface of the junction box 14, allowing for opening and closing of the clip 58. The open end 66 may face the distal end of the junction box 14 in some instances. In other instances, the open end 66 may face the proximal end of the junction box, if desired.

The second end 66 may include a tab curved away from the outer surface of the junction box 14 to allow for a user to grip and manually open the clip 58, or to allow for a wire, catheter, cord or the like to slide under the second end 66 and into the clip 58. In some instances, the clip 58 may be used to secure the junction box 14 to a bed sheet, a drape, a covering, a gown, a strap, a pole, a bed rail, or the like. The proximal curved portion may be the same or a different material as the rest of the clip 58. Proximal of the tab at the second end 66 of the clip 58, there may be a recess or bump 64 where the clip 58 is biased to touch the outer surface of the junction box 14. The junction box 14 may have a mating recess or bump 68 configured to receive the clip assembly bump 64. In one example, the clip 58 may include a bump 64 configured to nest within a recess 68 of the junction box 14 (e.g., a convex bump 64 configured to nest within a concave recess 68). In another example, the junction box 14 may include a bump configured to nest within a recess 68 of the clip 58 (e.g., a convex bump configured to nest within a concave recess). As the bumps/recesses 64 and 68 nest together, they prevent anything which may be retained between the clip 58 and the junction box 14, e.g., cord, elongate member, bed sheet, drape, covering, gown, strap, pole, bed rail or the like, from sliding out of the second end 66. There may be any number of bumps/recesses 64, 68 along the length of the clip assembly which may assist in coiling an elongate shaft of a catheter and/or passing a cord through the clip 58 multiple times.

With continued reference to FIG. 3, the area extending between the bumps/recesses 64, 68 and the first curved end 62 of the clip assembly 58 may include a space 82 between the clip 58 and the junction box 14. The clip 58 may be sized and shaped such that the space 82 can retain a catheter, wire, cord or the like (e.g., such as a coiled portion of a catheter, wire, cord, etc.) without pinching flattening, or otherwise affecting the retained object between a surface of the clip 58 facing the junction box 14 and the outer surface of the junction box 14. The clip 58 may be sized and shaped such that the space 82 may accommodate a bed sheet, a drape, a covering, a gown, a strap, a pole, a bed rail, or the like when disposed therein, holding the junction box 14 in place while the percutaneous circulatory support device 10 is in use. In some examples, the clip 58 may be oriented such that the second end 66 is facing a proximal end of the junction box 14 toward the cord 22 (FIG. 1), or the distal end of the junction box 14 toward the elongate member 12 (FIG. 1). There may be two clip assemblies, each having an opening facing the distal end, proximal end, or opposite ends of the junction box 14. Said differently, there may be any number of clips 58 disposed on the junction box 14, and the clips may be arranged to face any direction. In some examples, the clip 58 may be configured to rotate relative to the junction box 14 (e.g., be connected to the junction box 14 via a pivotable or rotatable connection), such that a physician can orient the opening end of the clip 66 to face any direction relative to the junction box 14. For example, the opening end of the clip 66 may face the distal end of the junction box 14 or the opening end of the clip 66 may face the proximal end of the junction box 14, as desired.

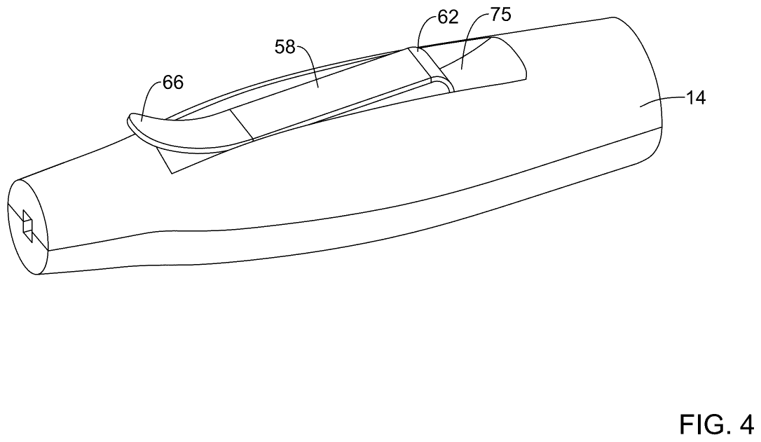

Alternatively, or additionally to any of the embodiments previously discussed, the clip 58 may be embedded within the junction box 14 (e.g., recessed below the outer surface of the junction box 14) as shown in FIG. 4. Recessing the clip 58 within the junction box 14 may reduce the potential for the clip 58 to catch or snag on anything while the percutaneous circulatory support device 10 is in use. In some examples, the clip 58 may be recessed within a channel 75 in the junction box 14. The channel 75 may be a recessed portion of the junction box 14, having side walls and a base wall extending between the side walls. The clip 58 may be positioned within the channel 75 between the side walls of the channel 75, with the first curved portion 62 connected to or otherwise extending from the base wall of the channel 75.

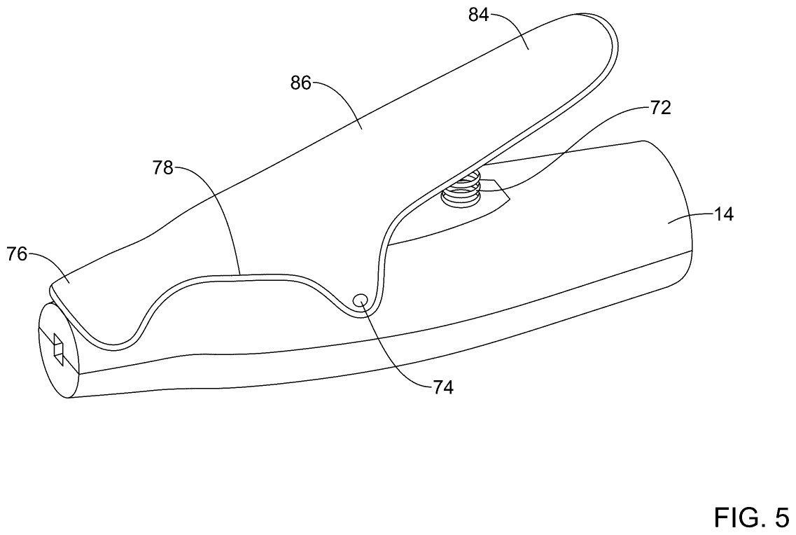

Additionally, FIG. 5 illustrates that the clip assembly may be made of a non-flexible material pivotably connected to the junction box 14 with one or more hinges 74 and one or more springs 72. The clip 86 may be designed such that it is biased to a closed position wherein the first end 76 is touching the outer surface of the junction box 14. In one embodiment, the spring 72 extends from the outer surface of the junction box 14, to the second end 84 of the clip assembly. The spring 72 has a bias which urges the second end 84 of the clip 86 away from the outer surface of the junction box 14, thereby pushing the first end 76 toward the outer surface of the junction box 14 to touch the junction box 14 as the clip 86 pivots about its pivot axis. The clip 86 may be pivotably attached to the junction box 14 with a hinge or pins 74 between the first and second ends 84 and 76. A physician may press down on the second end 84 of the clip 86, thereby opening the first end 76 to allow for inserting a wire, a cord, a bed sheet, a drape, a covering, a gown, a strap, a pole, a bed rail, or the like to be retained. In other embodiments, the clip 86 may include a torsion spring arranged with the hinge or pins 74 to bias the first end 76 of the clip 86 toward and into engagement with the outer surface of the junction box 14.

When the clip 86 is in the closed position, the space between the first end 76 of the clip 86 and the hinges 74 forms a gap between the clip 86 and the junction box 14 within which a catheter, wire, cord or the like can be retained (e.g., such as a coiled portion of a catheter, wire, cord, etc.). The portion 78 of the clip 86 extending between the first end 76 and hinges 74 may be sized and shaped to retain a catheter, wire, cord or the like (e.g., such as a coiled portion of a catheter, wire, cord, etc.) without pinching flattening, or otherwise affecting the retained object. In some embodiments, the portion 78 of the clip 86 extending between the first end 76 and hinges 74 may be sized and shaped to fit over a strap, a pole, or bed rail, holding the junction box 14 in place while the percutaneous circulatory support device is in use. In some embodiments, the portion 78 of the clip 86 extending between the first end 76 and hinges 74 may be sized and shaped to clasp a bed sheet, a drape, a covering, a gown, or the like, holding the junction box 14 in place while the percutaneous circulatory support device is in use. In some embodiments, the portion 78 of the clip 86 which forms the gap between the clip 86 and the outer surface of the junction box 14 may be shaped or contoured to facilitate gripping the object placed therebetween. In some embodiments, the portion 78 of the clip 86 which forms the gap between the clip 86 and the outer surface of the junction box 14, may include one or more recesses and/or bumps 64 as shown in FIG. 3, to allow for winding a cord through the clip 86 multiple times.

The materials that can be used for the various components of the circulatory support device (and/or other systems or components disclosed herein) and the various elements thereof disclosed herein may include those commonly associated with medical devices. For simplicity purposes, the following discussion makes reference to the circulatory support device (and variations, systems or components disclosed herein). However, this is not intended to limit the devices and methods described herein, as the discussion may be applied to other elements, members, components, or devices disclosed herein.

In some embodiments, the circulatory support device (and variations, systems or components thereof disclosed herein) may be made from a metal, metal alloy, ceramics, zirconia, polymer (some examples of which are disclosed below), a metal-polymer composite, 3D printed materials such as acrylonitrile butadiene styrene (ABS), combinations thereof, and the like, or other suitable material. Some examples of suitable metals and metal alloys include stainless steel, such as 444V, 444L, and 314LV stainless steel; mild steel; nickel-titanium alloy such as linear-clastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-clastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-clastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially clastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-clastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about-60 degrees Celsius (C) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-clastic plateau and/or flag region. For example, across a broad temperature range, the linear clastic and/or non-super-clastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-clastic alloy, for example a super-elastic nitinol can be used to achieve desired properties.

In at least some embodiments, portions or all of the circulatory support device (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the circulatory support device (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the circulatory support device (and variations, systems or components thereof disclosed herein) to achieve the same result.

In some embodiments, the circulatory support device (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In some embodiments, the circulatory support device (and variations, systems or components thereof disclosed herein) may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A percutaneous circulatory support device comprising:
a catheter including an elongate shaft and a percutaneous blood pump positioned at a distal end of the elongate shaft;
a junction box having a proximal end and a distal end;
wherein the junction box is secured to the elongate shaft proximal of the percutaneous blood pump such that the elongate shaft extends between the junction box and the percutaneous blood pump; and
a clip assembly disposed on an outer surface of the junction box.

2. The device of claim 1, wherein the clip assembly further comprises:
a base end portion attached to and extending radially outward from the outer surface of the junction box;
a free end portion biased to touch the outer surface of the junction box; and
a central portion extending between the base end portion and the free end portion.

3. The device of claim 2, wherein the base end portion comprises a preformed curved portion.

4. The device of claim 2, wherein the clip assembly is comprised of a flexible material.

5. The device of claim 2, wherein the junction box further comprises a recess along the outer surface of the junction box, disposed under the free end portion of the clip assembly.

6. The device of claim 5, wherein the clip assembly includes a bump configured to nest within the recess of the junction box.

7. The device of claim 2, wherein the central portion of the clip assembly is spaced apart from the junction box defining a space between the outer surface of the junction box and the central portion of the clip assembly.

8. The device of claim 7, wherein the clip assembly is configured to retain the elongate shaft within the space between the outer surface of the junction box and central portion of the clip assembly.

9. The device of claim 7, wherein the clip assembly is configured to retain a bed sheet, a drape, a covering, a gown, a strap, a pole, or a bed rail within the space between the outer surface of the junction box and the central portion of the clip assembly.

10. The device of claim 1, wherein the junction box further includes a channel disposed on the outer surface of the junction box.

11. The device of claim 10, wherein the clip assembly is disposed within the channel.

12. The device of claim 11, wherein a free end portion of the clip assembly is configured to curve or extend radially outward at an angle from the junction box beyond the channel.

13. The device of claim 1, wherein the clip assembly further comprises:
a central portion pivotably coupled to the junction box;
a first end portion extending from the central portion;
a second end portion extending from the central portion opposite the first end portion;
a spring biasing the second end portion against the outer surface of the junction box.

14. The device of claim 13, wherein the first end portion is spaced away from the outer surface of the junction box when the second end portion is urged against the outer surface of the junction box.

15. The device of claim 14, wherein the second end portion is configured to be moved away from the outer surface of the junction box as the first end portion is moved toward the outer surface of the junction box.

16. A percutaneous circulatory support device comprising:
a catheter including an elongate shaft and a percutaneous blood pump positioned at a distal end of the elongate shaft;
a junction box having a proximal end and a distal end;
wherein the elongate shaft extends distally from the distal end of the junction box to the percutaneous blood pump;
a clip assembly disposed on an outer surface of the junction box, the clip assembly comprising:
a base end portion attached to and extending radially outward from the outer surface of the junction box;
a free end portion biased to touch the outer surface of the junction box; and
a central portion extending between the base end portion and the free end portion;
wherein the clip assembly is comprised of a flexible material such that the free end portion can be deflected away from the outer surface of the junction box.

17. The device of claim 16, wherein the junction box further comprises a recess along the outer surface of the junction box, wherein the free end portion of the clip assembly extends into the recess.

18. The device of claim 17, wherein the clip assembly is spaced apart from the junction box between the base end portion and the free end portion to define a space therebetween, wherein the clip assembly is configured to retain the elongate shaft within the space between the junction box and the clip assembly.

19. A percutaneous circulatory support device comprising:
a catheter including an elongate shaft and a percutaneous blood pump positioned at a distal end of the elongate shaft;
a junction box having a proximal end and a distal end;
wherein the elongate shaft extends distally from the distal end of the junction box to the percutaneous blood pump;
a clip assembly pivotably coupled to the junction box, wherein the clip assembly comprises:
a central portion pivotably coupled to the junction box;
a first end portion extending from the central portion;
a second end portion extending from the central portion opposite the first end portion;
a spring biasing the second end portion against the outer surface of the junction box.

20. The device of claim 19, wherein the second end portion is configured to be moved away from the outer surface of the junction box as the first end portion is moved toward the outer surface of the junction box.
